(19) United States
(12) Patent Application Publication
(10) Pub. No.: US 2013/0066331 A1
(43) Pub. Date: Mar. 14, 2013

(54) TAPERED, CURVED STYLETS FOR INSERTING SPINAL CORD MODULATION LEADS AND ASSOCIATED SYSTEMS AND METHODS

(71) Applicants: Yougandh Chitre, Santa Clara, CA (US); Andre B. Walker, Monte Sereno, CA (US); Vivek Sharma, San Ramon, CA (US); Ellen Moore, Menlo Park, CA (US)

(72) Inventors: Yougandh Chitre, Santa Clara, CA (US); Andre B. Walker, Monte Sereno, CA (US); Vivek Sharma, San Ramon, CA (US); Ellen Moore, Menlo Park, CA (US)

(73) Assignee: Nevro Corporation, Menlo Park, CA (US)

(21) Appl. No.: 13/232,714

(22) Filed: Sep. 14, 2011

(51) Int. Cl. A61B 19/00; A61N 1/36

(52) U.S. Cl. 606/129

(57) ABSTRACT

The present technology is directed generally to tapered, curved stylets for inserting spinal cord modulation leads, and associated systems and methods. In some embodiments, the stylet includes a handle and a shaft the shaft having a proximal portion adjacent to the handle, a distal portion adjacent to the proximal portion, and a rounded tip. The proximal portion can be elongated along a longitudinal axis and can have a generally constant diameter. The distal portion can have a tapered section with a diameter that decreases in a distal direction, and a pre-set curve with respect to the longitudinal axis. The rounded tip can have a diameter greater than or equal to the smallest diameter of the distal portion.

### TECHNICAL FIELD

The present technology is directed generally to tapered, curved stylets for inserting and positioning spinal cord modulation leads.

### BACKGROUND

Neurological stimulators have been developed to treat pain, movement disorders, functional disorders, spasticity, cancer, cardiac disorders, and various other medical conditions. Implantable neurological stimulation systems generally have an implantable pulse generator and one or more leads that deliver electrical pulses to neurological tissue or muscle tissue. For example, several neurological stimulation systems for spinal cord stimulation (SCS) have cylindrical leads that include a lead body with a circular cross-sectional shape and multiple conductive rings spaced apart from each other at the distal end of the lead body. The conductive rings operate as individual electrodes or contacts and the SCS leads are typically implanted either surgically or percutaneously through a large needle inserted into the epidural space, often with the assistance of a stylet.

Once implanted, the pulse generator applies electrical pulses to the electrodes, which in turn modify the function of the patient's nervous system, such as by altering the patient's responsiveness to sensory stimuli and/or altering the patient's motor-circuit output. The electrical pulses can generate sensations that mask or otherwise alter the patient's sensation of pain. For example, in many cases, patients report a tingling or paresthesia that is perceived as more pleasant and/or less uncomfortable than the underlying pain sensation. In other cases, the patients can report pain relief without paresthesia or other sensations.

In any of the foregoing systems, it is important for the practitioner to accurately position the stimulator in order to provide effective therapy. With varying patient anatomies and tight spaces in which to navigate, practitioners often must frequently change out the stylet during implantation in order to accurately place the lead. Insertion and withdrawal forces during stylet change can damage the lead or the contact site, for example, or pose an inconvenience for the practitioner. Accordingly, the process of placing the lead can be difficult. As a result, there exists a need for a stylet which provides for simplified lead navigation and ease of insertion and removal from the lead.

### BRIEF DESCRIPTION OF THE DRAWINGS

FIG. 1A is a partially schematic illustration of an implantable spinal cord modulation system positioned at a patient's spine to deliver therapeutic signals in accordance with several embodiments of the present disclosure.

FIG. 1B is a partially schematic, cross-sectional illustration of a patient's spine, illustrating representative locations for an implanted lead in accordance with embodiments of the disclosure.

FIG. 2 is a side cross-sectional illustration of a stylet configured in accordance with embodiments of the disclosure.

FIG. 3 is an end cross-sectional illustration of the stylet taken substantially along line 3-3 of FIG. 2.

FIG. 4 is a partially schematic, enlarged illustration of a representative signal delivery device configured in accordance with embodiments of the disclosure.

### DETAILED DESCRIPTION

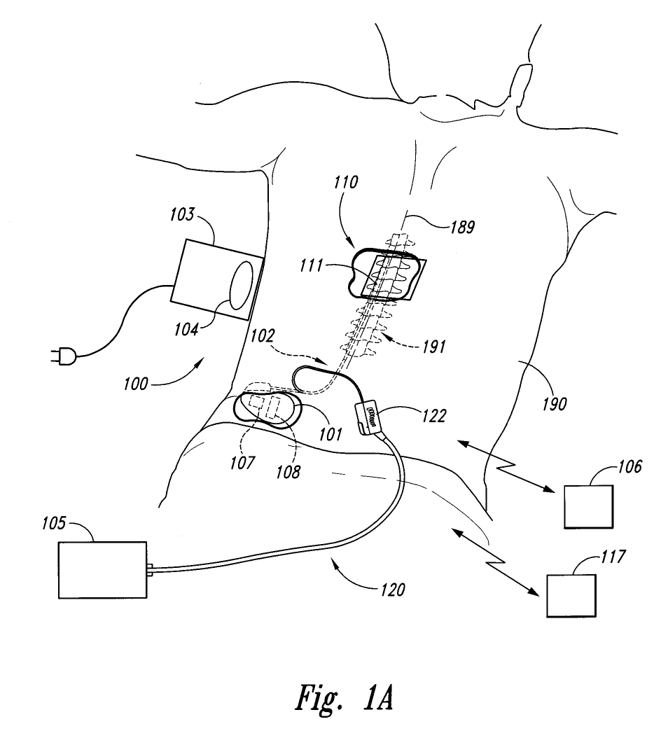

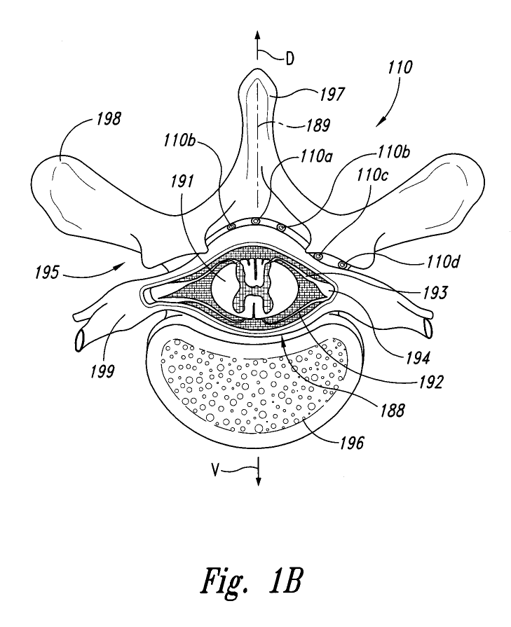

The present technology is directed generally to stylets for inserting and positioning spinal cord modulation leads and associated systems and methods. In at least some contexts, a portion of the stylet is tapered and curved. The tapered, curved portion of the stylet eases navigation through the patient anatomy surrounding a spinal modulation site and reduces excessive insertion and withdrawal forces when changing the stylet. The stylet can include a rounded tip that further reduces the risk of puncturing the lead body. In other embodiments, the technology and associated methods can have different configurations, components, and/or procedures. Still other embodiments may eliminate particular components and/or procedures. A person of ordinary skill in the relevant art, therefore, will understand that the present technology which includes associated devices, systems, and procedures may include other embodiments with additional elements or steps, and/or may include other embodiments without several of the features or steps shown and described below with reference to FIGS. 1A-4. Several aspects of overall systems in accordance with the disclosed technology are described with reference to FIGS. 1A and 1B, and features specific to stylets are then discussed with reference to FIGS. 2-4.

FIG. 1A schematically illustrates a representative patient system 100 for providing relief from chronic pain and/or other conditions, arranged relative to the general anatomy of a patient's spinal cord 191. The overall patient system 100 can include a signal delivery device 110, which may be implanted within a patient 190, typically at or near the patient's spinal cord midline 189, and coupled to a pulse generator 101. The signal delivery device 110 carries features for delivering therapy to the patient 190 after implantation. The pulse generator 101 can be connected directly to the signal delivery device 110, or it can be coupled to the signal delivery device 110 via a signal link 102 (e.g., an extension). In a further representative embodiment, the signal delivery device 110 can include one or more elongated lead(s) or lead body or bodies 111. As used herein, the terms “lead” and “lead body” include any of a number of suitable substrates and/or support members that carry devices for providing therapy signals to the patient 190. For example, the lead or leads 111 can include one or more electrodes or electrical contacts that direct electrical signals into the patient's tissue, such as to provide for patient relief. In other embodiments, the signal delivery device 110 can include structures other than a lead body (e.g., a paddle) that also direct electrical signals and/or other types of signals to the patient 190.

The pulse generator 101 can transmit signals (e.g., electrical signals) to the signal delivery device 110 that up-regulate (e.g., stimulate or excite) and/or down-regulate (e.g., block or suppress) target nerves. As used herein, and unless otherwise noted, the terms “modulate” and “modulation” refer generally to signals that have either type of the foregoing effects on the target nerves. The pulse generator 101 can include a machine-readable (e.g., computer-readable) medium containing instructions for generating and transmitting suitable therapy signals. The pulse generator 101 and/or other elements of the system 100 can include one or more processors 107, memories 108 and/or input/output devices. Accordingly, the process of providing modulation signals, providing guidance information for locating the signal delivery device 110, and/or executing other associated functions can be performed by computer-executable instructions contained by computer-readable media located at the pulse generator 101 and/or other system components. The pulse generator 101 can include multiple portions, elements, and/or subsystems (e.g., for directing signals in accordance with multiple signal delivery parameters), carried in a single housing, as shown in FIG. 1A, or in multiple housings.

In some embodiments, the pulse generator 101 can obtain power to generate the therapy signals from an external power source 103. The external power source 103 can transmit power to the implanted pulse generator 101 using electromagnetic induction (e.g., RF signals). For example, the external power source 103 can include an external coil 104 that communicates with a corresponding internal coil (not shown) within the implantable pulse generator 101. The external power source 103 can be portable for ease of use.

During at least some procedures, an external programmer 105 (e.g., a trial modulator) can be coupled to the signal delivery device 110 during an initial procedure, prior to implanting the pulse generator 101. For example, a practitioner (e.g., a physician and/or a company representative) can use the external programmer 105 to vary the modulation parameters provided to the signal delivery device 110 in real time, and select optimal or particularly efficacious parameters. These parameters can include the location from which the electrical signals are emitted, as well as the characteristics of the electrical signals provided to the signal delivery device 110. In a typical process, the practitioner uses a cable assembly 120 to temporarily connect the external programmer 105 to the signal delivery device 110. The practitioner can test the efficacy of the signal delivery device 110 in an initial position. The practitioner can then disconnect the cable assembly 120 (e.g., at a connector 122), reposition the signal delivery device 110, and reapply the electrical modulation. This process can be performed iteratively until the practitioner obtains the desired position for the signal delivery device 110. Optionally, the practitioner may move the partially implanted signal delivery element 110 without disconnecting the cable assembly 120.

After a trial period with the external programmer 105, the practitioner can implant the implantable pulse generator 101 within the patient 190 for longer term treatment. The signal delivery parameters provided by the pulse generator 101 can still be updated after the pulse generator 101 is implanted, via a wireless physician's programmer 117 (e.g., a physician's remote) and/or a wireless patient programmer 106 (e.g., a patient remote). Generally, the patient 190 has control over fewer parameters than does the practitioner.

FIG. 1B is a cross-sectional illustration of the spinal cord 191 and an adjacent vertebra 195 (based generally on information from Crossman and Neary, “Neuroanatomy,” 1995 (published by Churchill Livingstone)), along with multiple signal delivery devices 110 (shown as signal delivery devices 110a-d) implanted at representative locations. For purposes of illustration, multiple signal delivery devices 110 are shown in FIG. 1B implanted in a single patient. In actual use, any given patient will likely receive fewer than all the signal delivery devices 110 shown in FIG. 1B.

The spinal cord 191 is situated within a vertebral foramen 188, between a ventrally-located ventral body 196 and a dorsally-located transverse process 198 and spinous process 197. Arrows V and D identify the ventral and dorsal directions, respectively. The spinal cord 191 itself is located within the dura mater 199, which also surrounds portions of the nerves exiting the spinal cord 191, including the ventral roots 192, dorsal roots 193 and dorsal root ganglia 194. In one embodiment, a single first signal delivery device 110a is positioned within the vertebral foramen 188, at or approximately at the spinal cord midline 189. In another embodiment, two second signal delivery devices 110b are positioned just off the spinal cord midline 189 (e.g., about 1 mm. offset) in opposing lateral directions so that the two signal delivery devices 110b are spaced apart from each other by about 2 mm. In still further embodiments, a single signal delivery device or pairs of signal delivery devices can be positioned at other locations, e.g., at the dorsal root entry zone as shown by a third signal delivery device 110c, or at the dorsal root ganglia 194, as shown by a fourth signal delivery device 110d.

In any of the foregoing embodiments, it is important that the signal delivery device 110 and in particular, the therapy or electrical contacts of the device, be placed at a target location that is expected (e.g., by a practitioner) to produce efficacious results in the patient when the device 110 is activated. The following disclosure describes techniques and systems for simplifying the process of placing contacts via which to deliver neural modulation signals to the patient.

FIG. 2 is a partially schematic, cross-sectional side view of a stylet 161 that can be temporarily coupled to a lead to support the lead as it is inserted into the patient's epidural space in accordance with embodiments of the present disclosure. The stylet 161 can include a handle 163 that can be fixedly or removably attached to a shaft 162 having a distal portion 165 and a proximal portion 166. The handle 163 can be made of any number of suitable biocompatible materials. In one embodiment, for example, the handle 163 comprises a thermoplastic such as acrylonitrile butadiene styrene (ABS). The proximal portion 166 of the shaft 162 can be elongated along a longitudinal axis L and can have a generally uniform diameter Dₛ. The distal portion 165 of the shaft 162 can include a tapered section 164 having a distally-decreasing diameter Dₜ less than the diameter Dₛ of the proximal portion 166. For example, in some embodiments, the diameter Dₜ of the tapered section 164 decreases from the proximal portion diameter Dₛ to a minimum diameter Dₘ that is from about 40% to about 99% of the proximal portion diameter Dₛ. In a particular embodiment, the minimum diameter Dₘ can be 88% of the proximal portion diameter Dₛ. In particular embodiments, the diameter Dₛ can range from about 0.009 inch to about 0.020 inch while the minimum diameter Dₘ of the tapered section 164 can range from about 0.008 inch to about 0.016 inch. In further particular embodiments, the diameter Dₛ of the proximal portion 166 is from about 0.012 inch to about 0.014 inch while the minimum diameter Dₘ of the tapered section 164 is from about 0.011 inch to about 0.014 inch. The slope or taper ratio of the taper between the proximal portion 166 and the minimum diameter Dₘ of the tapered section 164 can be constant or can vary along the length of the tapered section 164. As used herein, the taper ratio refers to the change in stylet diameter divided by the taper length. Depending upon the embodiment, the taper ratio can be constant around the circumference of the tapered section 164 (e.g., the tapered section 164 can slope relative to the longitudinal axis L at a constant rate around the circumference of a cross-section of the tapered section) or the slope can vary around the circumference (e.g., the tapered section 164 can have a greater slope relative to the longitudinal axis at one circumferential location around the cross-section than at another circumferential location). In some cases, the slope can be zero at one or more circumferential locations.

The tapered section 164 can have a length Lₜ of from about 0.20 inch to about two inches, while the stylet 161 can have a total length Lₛ of from about five inches (approximately 12 centimeters) to about 40 inches (approximately 100 centimeters). In one embodiment, the tapered section 164 has a length Lₜ of from about 0.45 inch to about 0.75 inch and the stylet 161 has a length Lₛ of from about 12 inches (approximately 30 centimeters) to about 28 inches (approximately 70 centimeters). Accordingly, in several embodiments, the length Lₜ of the tapered section 164 is a fraction of the total length Lₛ of the stylet 161. For example, in some embodiments, the length Lₜ of the tapered section 164 can be from about 0.5% to about 11% of the total stylet length Lₛ, and in a particular embodiment, about 2%. The taper ratio over the extent of the tapered section 164 can be from about 0.001 to about 0.055, and in a particular embodiment, about 0.003. As will be described in further detail later, the characteristics of the tapered section 164 can be selected to ease the task of removing the stylet 161 from the lead without compromising the practitioner's ability to position the lead with the stylet 161.

The distal portion 165 can have a pre-set curve 167 that extends through a deflection angle a relative to the longitudinal axis L. The deflection angle a can range from about 5° to about 40° with respect to the longitudinal axis L. In one embodiment, the deflection angle a can range from about 15° to about 30° with respect to the longitudinal axis L. In other embodiments, the distal portion 165 can curve in multiple planes, e.g., to form a partially spiral shape. The pre-set curve 167 can occupy all or a portion of the length of the tapered section 164 and/or the shaft 162. The pre-set curve 167 can allow the practitioner to readily redirect the lead during an implant procedure, as will be described in further detail later. In still further embodiments, the stylet 161 can be generally straight along its length Lₛ, with no pre-set curve.

The stylet 161 can include a rounded tip 168 on the distal portion 165 to reduce the likelihood for the stylet 161 to penetrate through the lead. In some embodiments, the rounded tip 168 has a diameter from about 0.011 inch to about 0.014 inch. The rounded tip 168 can have a diameter greater than the smallest diameter Dₘ of the tapered section 164, but less than the diameter Dₛ of the proximal portion 166. The rounded tip 168 can be soldered, welded, or otherwise affixed to the shaft 162, or the rounded tip 168 can be integrally formed with the shaft 162. In some embodiments, the rounded tip 168 can include a material providing radiopacity or enhanced radiopacity relative to the shaft 192. Such materials include palladium, tungsten, tantalum, gold, platinum, iridium, and alloys thereof. In one embodiment, for example, the tip 168 comprises a platinum-iridium alloy, such as Pt₉₀Ir₁₀.

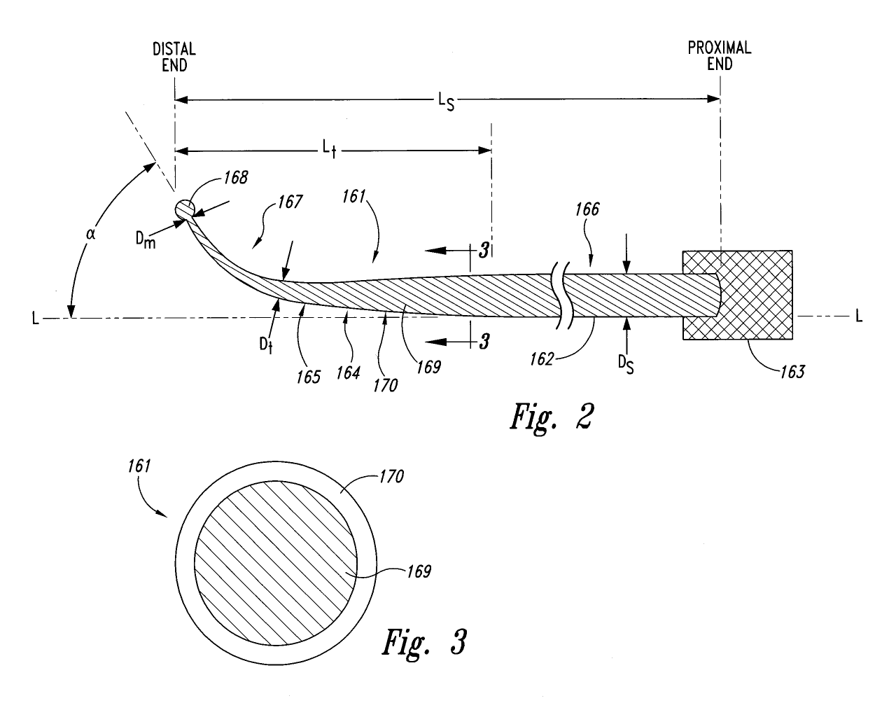

The stylet 161 can be made primarily of stainless steel or other suitable biocompatible materials (including, e.g., titanium, nickel titanium and other metals and alloys thereof) having comparable mechanical properties. In some embodiments, the stylet 161 or a portion of the stylet 161 has a stiffness greater than a stiffness of the lead 111 (FIG. 1A) in which it is inserted. The stiffness of the stylet 161 indicates a resistance to bending away from the longitudinal axis L. As described in further detail below with reference to FIG. 3, in some embodiments, at least a portion of the stylet 161 can be coated with a layer of polytetrafluoroethylene (PTFE) or another suitable fluoropolymer. Accordingly, the stylet can include an inner core 169 and an outer coating 170.

FIG. 3 is a cross-sectional illustration of the stylet 161, taken substantially along line 3-3 of FIG. 2. In the illustrated embodiment, the coating 170 surrounds the entire circumference of the inner core 169. In other embodiments, the coating 170 covers only a portion of the outer circumference of the inner core 169. Furthermore, the coating 170 can cover all or only a portion of the length Lₛ of the stylet 161. For example, in one embodiment, only the proximal portion 166 is coated, while the tapered section 164 is uncoated. In another embodiment, both the proximal portion 166 and the tapered section 164 are coated. For example, the coating 170 can be applied to both the proximal portion 166 and the tapered section 164 and then ground off from at least a portion of the tapered section 164 so that the tapered section 164 is no longer coated. The coating 170 illustrated in FIG. 3 is not necessarily to scale. The coating 170 can have a thickness from about 0.0001 inch to about 0.002 inch, and in one embodiment, has a thickness from about 0.0001 inch to about 0.0005 inch. In other embodiments, the stylet 161 can have other types of coatings 170 or no coating at all. In any of these embodiments, the core 169 has a first coefficient of friction and the coating 170 has a second coefficient of friction less than the first. Accordingly, the coating 170 can facilitate inserting and removing the stylet 161 by reducing the sliding friction between the stylet 161 and the lead 111.

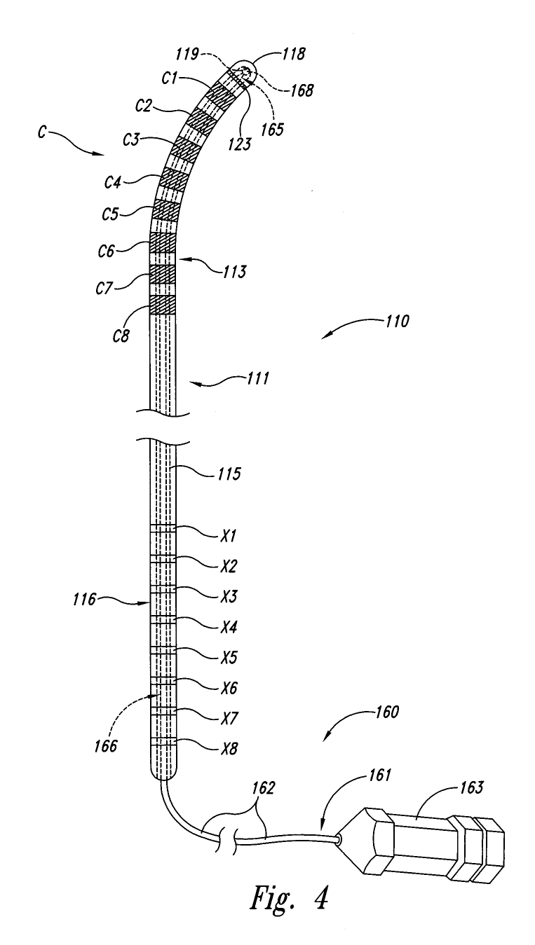

FIG. 4 is a partially schematic illustration of a representative signal delivery device 110 that includes a lead 111 having a distal region 113 that carries a plurality of ring-shaped therapy contacts or electrical contacts C positioned to deliver therapy signals to the patient when the lead 111 is implanted. In a representative embodiment, the lead 111 includes eight therapy or electrical contacts C, identified individually as contacts C1, C2, C3 . . . C8. The lead 111 includes internal wires or conductors (not visible in FIG. 4) that extend between the contacts C at or near the distal region 113 of the lead 111, and corresponding connection contacts X (shown as X1, X2, X3 . . . X8) positioned at or near a proximal region 116 of the lead 111. Contacts C and X can be made of any biocompatible metal such as titanium, a noble metal such as platinum or iridium, or alloys thereof. In some embodiments, the contacts C and X can be coated with materials to improve contact performance or increase the surface area of the contacts C and X. These materials can include, for example, platinum black, titanium nitride, iridium oxide, or other materials having generally similar material properties. After implantation, the connection contacts X are connected to the external programmer 105 or to the implanted pulse generator 101 discussed above with reference to FIG. 1A.

The lead 111 terminates at a lead distal end or distal end portion 118. The lead distal end 118 can be made of the same material as the rest of the lead 111 or can be made of a separate material or component. In some embodiments, the lead distal end 118 includes a biocompatible material such as silicone, silicone-polyurethane co-polymers, polyurethanes and elastomers thereof (such as Pellethane® made by The Lubrizol Corp., of Wickliffe, Ohio). In some embodiments, the lead distal end 118 can include a radiopaque portion 123 made of, for example, titanium dioxide or barium sulfate, to aid in positioning the lead 111 via fluoroscopy or another suitable visualization technique.

During implantation, the stylet 161 is temporarily coupled to the lead 111 to support the lead 111 as it is inserted into the patient. For example, the shaft 162 of the stylet 161 is slideably and releasably inserted (via the handle 163) into an axially-extending opening (lumen 115) in the lead 111. In some embodiments, the lumen 115 has a diameter from about 0.015 inch to about 0.030 inch. The ratio of the minimum diameter Dₘ of the tapered section 164 to the lumen diameter can be from about 0.3 to about 0.99, and in a particular embodiment, about 0.6.

The stylet rounded tip 168, when inserted into the lumen 115, is restricted/prevented from extending past the lead distal end portion 118. Rather, when the practitioner moves the stylet 161 through lead lumen 115 to the distal end portion 118, the stylet rounded tip 168 will eventually abut a stylet stop 119 located at the terminus of lumen 115 at the distal end portion 118. The stylet stop 119 can prevent the stylet 161 from further distal progression. In some embodiments, the roundness of the tip 168 provides less pressure on the lead and/or the dura mater of the patient than would a sharp tip, such that when the rounded tip 168 contacts the stylet stop 119 of the lead 111 or (less likely) the patient's dura mater, the rounded tip 168 is unlikely to perforate these surfaces. In some embodiments, the lead 111 is positioned in a catheter (not shown in FIG. 4). The catheter is inserted into the patient's body. The lead 111 is deployed from the catheter using the stylet 161. The lead 111 and the stylet 161 are then moved together to position the lead 111 proximate to a spinal modulation site.

The shaft 162 of the stylet 161 is generally flexible but more rigid than the lead 111, and can provide added column stiffness to the lead while the stylet 161 is inserted the lead 111. This can allow the practitioner to more readily deploy, support and control the lead 111 and its position during implantation. Under the application of sufficient bending force, the distal portion 165 of the stylet 161 can bend in a resilient manner when pressure is applied to it, and can later resiliently return to any pre-set shape (e.g., the curve 167 shown in FIG. 2). In some clinical situations, the bending force applied to the stylet 161 does not cause the deflection angle α of the stylet distal portion 165 to appreciably change beyond its predetermined value. The curve 167 of the stylet 161 allows the practitioner to more easily make turns at or on the way to the spinal cord modulation site. To change the direction of the lead 111, the practitioner need only rotate the handle 163 (and thus the shaft 162) around the longitudinal axis L so that the distal end of the curve 167 points in a new direction.

After positioning the lead 111, the stylet 161 can be readily and freely removed from the lumen 115 by withdrawing the tapered distal end 165 away from the spinal cord modulation site and extracting the stylet 161 from the lumen 115. Because the stylet 161 has a tapered diameter Dₜ that is less than the diameter of the lumen 115, the stylet 161 is unlikely to get caught or stuck in the lumen 115. This reduces the risk that the practitioner will have to apply an excessive pushing or pulling force on the stylet 161 and the lead 111, which can accordingly reduce the risk of displacing the distal region 113 of the lead 111, damaging the lead 111, or injuring the patient. In some embodiments, at least one of an inner surface of the lumen 115 and an outer surface of the stylet 161 can include a material positioned to facilitate relative sliding and free separation between the surfaces. For example, a PTFE liner or the coating 170 described above in the context of the stylet 161 can be placed on the inner surface of the lead lumen, in addition to or in lieu of placing it on the stylet 161.

Unlike traditional cardiac stylets, which must be extremely flexible so as to avoid penetrating the wall of the right ventricle of the heart, stylets in accordance with embodiments of the present technology are comparatively stiff in order to provide the stability and strength needed to position spinal modulation leads. Long, limp cardiac stylets can rely on gravity for directing the lead downwardly during implantation. Spinal modulation leads, on the other hand, must be sufficiently rigid to allow the practitioner to steer the leads around patient muscle, bone, and/or scar tissue, while remaining yielding enough to limit the risk of damage to the lead. Embodiments of the stylets disclosed herein can provide the advantages of easy insertability into, and removability from, spinal modulation leads, and/or improved steering capability, and/or the ability to redirect the distal end via the proximal end without compromising the support provided to the lead.

From the foregoing, it will be appreciated that specific embodiments of the technology have been described herein for purposes of illustration, but that various modifications may be made without deviating from the technology. For example, stylets in accordance with some embodiments include more than one pre-set curve, alternate types of coatings, and/or a tapered portion that is more or less resiliently bendable than described above. Certain aspects of the technology described in the context of particular embodiments may be combined or eliminated in other embodiments. For example, in some embodiments the stylet may not be coated or the distal portion may not include a rounded tip. Additionally, the stylets disclosed herein can be used with leads having shapes or designs other than those specifically described above. For example, the stylets can be used with leads similar to those disclosed in the following patent applications, which are herein incorporated by reference in their entirety: U.S. application Ser. No. 12/765,747, filed Apr. 22, 2010 and titled SELECTIVE HIGH FREQUENCY SPINAL CORD MODULATION FOR INHIBITING PAIN WITH REDUCED SIDE EFFECTS, AND ASSOCIATED SYSTEMS AND METHODS; U.S. application Ser. No. 12/104,230, filed Apr. 16, 2008 and titled TREATMENT DEVICES WITH DELIVER-ACTIVATED INFLATABLE MEMBERS, AND ASSOCIATED SYSTEMS AND METHODS FOR TREATING THE SPINAL CORD AND OTHER TISSUES; U.S. application Ser. No. 12/468,688, filed May 19, 2009 and titled IMPLANTABLE NEURAL STIMULATION ELECTRODE ASSEMBLIES AND METHODS FOR STIMULATING SPINAL NEURAL SITES; U.S. application Ser. No. 12/129,078, filed May 29, 2008 and titled PERCUTANEOUS LEADS WITH LATERALLY DISPLACEABLE PORTIONS, AND ASSOCIATED SYSTEMS AND METHODS; U.S. application Ser. No. 12/765,805, filed Apr. 22, 2010 and titled SELECTIVE HIGH FREQUENCY SPINAL CORD MODULATION FOR INHIBITING PAIN WITH REDUCED SIDE EFFECTS, AND ASSOCIATED SYSTEMS AND METHODS, INCLUDING IMPLANTABLE LEADS; and U.S. application Ser. No. 12/562,892, filed Sep. 18, 2009 and titled COUPLING FOR IMPLANTED LEADS AND EXTERNAL STIMULATORS, AND ASSOCIATED SYSTEMS AND METHODS. Further, while advantages associated with certain embodiments have been described in the context of those embodiments, other embodiments may also exhibit such advantages and not all embodiments need necessarily exhibit such advantages to fall within the scope of the present technology. Accordingly, the present disclosure and associated technology can encompass other embodiments not expressly described or shown herein.

I/we claim:

1. A patient therapy device, comprising:
a patient-implantable lead having a proximal region and a distal region, at least one electrical contact disposed at or near the distal region, and a lumen formed inside the lead; and
a stylet having a shaft with a proximal portion having a proximal portion diameter, and a distal portion, the distal portion having a tapered section and a rounded tip, wherein the distal portion of the stylet is sized to be removably inserted into the lumen, the proximal portion of the shaft being elongated along a longitudinal axis, the tapered section having a distally decreasing diameter less than the proximal portion diameter, with a portion of the tapered section curving away from the longitudinal axis.

2. The patient therapy device of claim 1, wherein the stylet distal portion tapered section is resiliently flexible and tends toward a configuration in which it curves away from the longitudinal axis.

3. The patient therapy device of claim 1, wherein the stylet distal portion tapered section has a tapered section length from about 0.2 inch to about 1 inch and wherein the tapered section has a diameter that decreases to a minimum diameter of about 0.011 inch.

4. The patient therapy device of claim 1, wherein the stylet distal portion tapered section is curved from about 5° to about 40° with respect to the longitudinal axis.

5. The patient therapy device of claim 1, wherein the stylet proximal portion diameter is a generally uniform diameter.

6. The patient therapy device of claim 1, wherein the lead lumen has an inner surface, the stylet has an outer surface, and at least one of the inner surface of the lead lumen and the outer surface of the stylet includes a material positioned to facilitate relative sliding between the inner surface and the outer surface.

7. The patient therapy device of claim 1, wherein the lead has a first stiffness and the stylet has a second stiffness greater than the lead first stiffness.

8. The patient therapy device of claim 1, wherein the stylet distal portion tapered section has a taper ratio of from about 0.001 to about 0.055.

9. The patient therapy device of claim 1, wherein the stylet distal portion tapered section has a tapered section length, the stylet has a stylet length, and the patient therapy device has a ratio of the tapered section length to the stylet length of from about 0.005 to about 0.2.

10. The patient therapy device of claim 1, wherein the stylet distal portion tapered section has a tapered section minimum diameter, the lead lumen has a lumen diameter, and a ratio of the tapered section minimum diameter to the lumen diameter is from about 0.3 to about 0.99.

11. The patient therapy device of claim 1, wherein the stylet distal portion tapered section has a tapered section minimum diameter and a ratio of the tapered section minimum diameter to the stylet shaft proximal portion diameter is from about 0.4 to about 0.99.

12. The patient therapy device of claim 1, wherein the stylet distal portion tapered section has a tapered section minimum diameter, the lead lumen has a lumen diameter, and a ratio of the tapered section minimum diameter to the lumen diameter is about 0.6.

13. The patient therapy device of claim 1, wherein the stylet distal portion tapered section has a tapered section minimum diameter and a ratio of the tapered section minimum diameter to the stylet shaft proximal portion diameter is about 0.88.

14. The patient therapy device of claim 1, wherein the stylet distal portion rounded tip has a diameter greater than the smallest diameter of the stylet distal portion tapered section.

15. The patient therapy device of claim 1, wherein the stylet is made primarily of stainless steel.

16. The patient therapy device of claim 1, wherein at least a portion of the stylet is coated with a layer of fluoropolymer having a thickness from about 0.0001 inch to about 0.002 inch.

17. The patient therapy device of claim 1, wherein at least a portion of the proximal portion of the stylet shaft is coated with a layer of fluoropolymer and the stylet distal portion tapered section is not coated with a layer of fluoropolymer.

18. The patient therapy device of claim 1, further comprising a handle attached to the stylet shaft, and wherein:
the shaft proximal portion extends from the handle to the stylet distal portion tapered section;
the stylet distal portion tapered section extends from the proximal portion to the rounded tip;
the proximal portion has a first length; and
the tapered section has a second length that is at most 10% of the first length.

19. A stylet for positioning a spinal cord modulation lead in a patient, comprising:
a handle; and
a shaft, the shaft having:
a proximal portion adjacent to the handle, the proximal portion being elongated along a longitudinal axis and having a generally constant diameter;
a distal portion adjacent to the proximal portion, the distal portion comprising a tapered section having a diameter that decreases in a distal direction, and a pre-set curve with respect to the longitudinal axis; and
a rounded tip having a diameter greater than or equal to the smallest diameter of the distal portion.

20. The stylet of claim 19, wherein the shaft comprises a core material having a first coefficient of friction and a coating on the core material having a second coefficient of friction less than the core material coefficient of friction.

21. The stylet of claim 19, wherein the distal portion tapered section has a tapered section length, the shaft has a shaft length, and a ratio of the tapered section length to the shaft length is about 0.02.

22. The stylet of claim 19, wherein the distal portion tapered section has a taper ratio of 0.003.

23. The stylet of claim 19, wherein the tip of the shaft is angled at a value of from about 15° to about 30° with respect to the longitudinal axis.

24. The stylet of claim 19, wherein a tip of the shaft is angled at a value of from about 5° to about 40° with respect to the longitudinal axis.

25. The stylet of claim 19, wherein the shaft proximal portion diameter is from about 0.009 inch to about 0.020 inch.

26. A method for treating a patient, comprising:
inserting a catheter into the patient;
positioning a lead in the catheter, the lead carrying an electrode;
deploying the lead from the catheter by positioning the lead with a stylet so that the lead is proximate to a spinal modulation site, wherein the stylet has a shaft elongated along a longitudinal axis and a tapered distal end having a pre-set curve extending away from the longitudinal axis; and
withdrawing the stylet from the lead by moving the tapered distal end of the stylet proximally away from the lead while the lead remains at the spinal modulation site.

27. The method of claim 26, wherein deploying the lead comprises moving the lead and the stylet together to position the lead at the spinal modulation site.

28. The method of claim 26, further comprising preventing the stylet from penetrating through the lead by positioning a rounded tip of the stylet against a lumen inner wall of the lead.

29. The method of claim 26, wherein withdrawing the stylet from the lead comprises removing the stylet from a lumen of the lead, wherein the lumen has a diameter greater than a distal end diameter of the stylet.

30. The method of claim 26, further comprising delivering electrical modulation signals to the spinal modulation site via the electrodes carried by the lead.

31. The method of claim 26, further comprising positioning the stylet in a lumen of the lead before deploying the lead from the catheter.

32. The method of claim 26, wherein the lead has a lumen defined at least in part by lumen walls, and wherein withdrawing the stylet comprises freely withdrawing the stylet without the stylet sticking to the lumen walls.

33. The method of claim 26, wherein deploying the lead comprises positioning the pre-set curve to point in a first direction, and wherein the method further comprises redirecting the lead by rotating the stylet shaft around the longitudinal axis so that the pre-set curve points in a second direction different than the first direction.

34. A method for treating a patient, comprising:
inserting a catheter into the patient's body;
positioning a stylet in a lumen of a lead, the lead carrying an electrode, wherein the stylet has a shaft elongated along a longitudinal axis and a tapered distal end having a pre-set curve extending away from the longitudinal axis;
positioning the lead and stylet in the catheter;
moving the lead and the stylet together to deploy the lead from the catheter;
positioning the lead with the stylet so that the lead is proximate to a spinal modulation site, wherein positioning the lead includes rotating the stylet shaft to redirect the pre-set curve;
preventing the stylet from penetrating through the lead by positioning a rounded tip of the stylet against an end wall of a lumen in the lead;
withdrawing the stylet from the lead by moving the tapered distal end of the stylet proximally away from the end wall while the lead remains at the spinal modulation site, wherein the lumen has a diameter greater than a distal end diameter of the stylet and wherein the stylet is withdrawn without the stylet sticking to inner walls of the lumen; and
delivering electrical modulation to the spinal modulation site via the electrode.
